(19) United States
(12) Patent Application Publication
(10) Pub. No.: US 2009/0062709 A1
(43) Pub. Date: Mar. 5, 2009

(54) Knee support

(76) Inventor: Pang-Ching Chiang, Taipei (TW)

Correspondence Address: BACON & THOMAS, PLLC, 625 SLATERS LANE, FOURTH FLOOR, ALEXANDRIA, VA, 22314-1176, US

(21) Appl. No.: 11/892,978

(22) Filed: Aug. 29, 2007

(51) Int. Cl. A61F 5/00

(52) U.S. Cl. 602/26

(57) ABSTRACT

A knee support having a body, a ring, at least one fastening strap, two securing straps and a gap. The body has a fastening end, a securing end and an outer surface. The ring is mounted through the body. The at least fastening strap extends from the fastening end and has at least one fastener and is long enough to allow the fastening strap to be wrapped around a knee and cross over the ring. The at least one fastening strap helps to secure the ring and thus provides extra support and protection for a patella of the knee. The two securing straps extend from the securing end and each has at least one fastener. The gap is formed between the securing straps.

### BACKGROUND OF THE INVENTION

1. Field of the Invention

The present invention relates to a knee support, especially to a knee support with a patella-protecting ring.

2. Description of the Prior Art

A human knee is a joint comprising several bones including a patella and has a front. The patella, commonly referred to as a kneecap, is located at the front of the knee and is therefore frequently injured in a variety of sports.

With reference to FIGS. 10 to 12, an embodiment of a conventional knee support is used to protect knees from being injured and comprises a body (61), a ring (64), a fastening protrusion (62), two securing straps (63) and a gap.

The body (61) is a belt and has two ends, an outer surface, an inner surface, an upper edge, a lower edge and a width. The outer surface is fluffy. The width is defined as a distance between the upper edge and the lower edge.

The ring (64) is mounted through the outer surface of the body (61) between the two ends and has a central hole. The central hole is used to fit a patella of a knee. The fastening protrusion (62) extends from one end of the body (61) and has a fastener (621). The fastener (621) is a hook portion of a hook-and-loop connector corresponding to and being attached detachably to the outer surface of the body (61) to hold the body around the knee.

The two securing straps (63) extend from the other end of the body (61), are used to press the ring (64) to the patella of the knee and each has an end and at least one fastener (631). The gap is defined between the two securing straps (63). The fastening protrusion (62) extends through the gap and attaches to the outer surface. The fastener (631) is a hook portion of a hook-and-loop connector being attached to the end of the securing strap (63) and attached detachably to the body (61) to allow each securing strap (63) to cross over the ring (64) and press the ring (64) against the patella for support.

However, when using the conventional knee support, the ring (64) is not secured well by just the securing straps (63).

Furthermore, with the reference to FIGS. 13 and 14, in order to fit a larger knee size of a taller or stronger user, another embodiment of a conventional knee support has a body (71) having an enlarged width. When fastened with the attachment of the fastener (731) to the outer surface of the body, the securing straps (73) only cross over the ring (74) slightly. As a result, the securing straps (73) of the embodiment cannot effectively press the ring (74) to secure the ring (74) to the patella.

To overcome the shortcomings, the present invention provides a knee support to mitigate or obviate the aforementioned problems.

### SUMMARY OF THE INVENTION

The main objective of the invention is to provide a knee support.

The knee support in accordance with the present invention has a body, a ring, at least one fastening strap, two securing straps and a gap. The body has a fastening end, a securing end and an outer surface. The ring is mounted through the body. Each fastening strap extends from the fastening end and has at least one fastener and is long enough to allow the fastening strap to be wrapped around a knee and cross over the ring. The at least one fastening strap helps to secure the ring and thus provides extra protection for a patella of the knee. The two securing straps extend from the securing end and each has at least one fastener. The gap is formed between the securing straps.

Other objectives, advantages and novel features of the invention will become more apparent from the following detailed description when taken in conjunction with the accompanying drawings.

### BRIEF DESCRIPTION OF THE DRAWINGS

FIG. 1 is a front view of a first embodiment of a knee support in accordance with the present invention;

FIG. 2 is a perspective view of the knee support in FIG. 1 wherein the knee support is being fastened;

FIG. 3 is a front view of the knee support in FIG. 2, shown fastened, with straps and a ring shown in phantom lines;

FIG. 4 is a front view of a variation of the first embodiment of a knee support in accordance with the present invention;

FIG. 5 is a front view of the knee support in FIG. 4, shown fastened with a ring in phantom lines;

FIG. 6 is a front view of a second embodiment of a knee support in accordance with the present invention;

FIG. 7 is a front view of the knee support in FIG. 6 wherein the knee support is fastened with straps and the ring shown in phantom lines;

FIG. 8 is a front view of a variation of the second embodiment of a knee support in accordance with the present invention;

FIG. 9 is a front view of the knee support in FIG. 8 wherein the knee support is fastened, with straps and a ring shown in phantom lines;

FIG. 10 is a front view of an embodiment of a conventional knee support in accordance with the prior art;

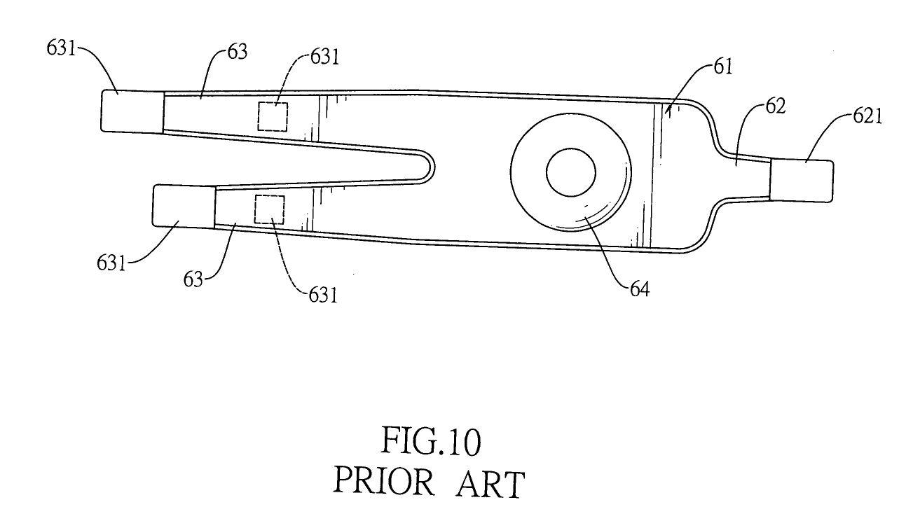

FIG. 11 is a perspective view of the knee support in FIG. 10 wherein the knee support is being fastened;

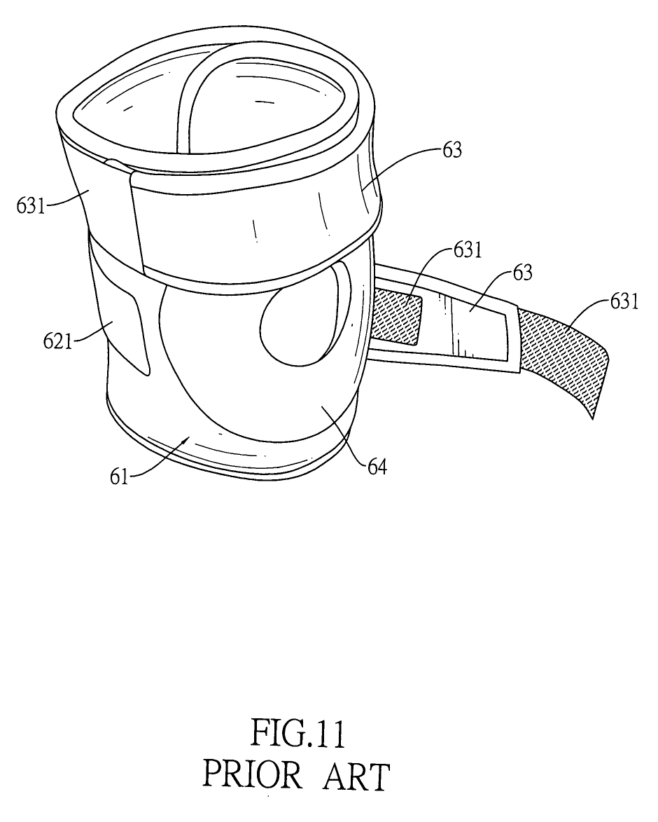

FIG. 12 is a front view of the knee support in FIG. 11, shown fastened with a ring shown in phantom lines;

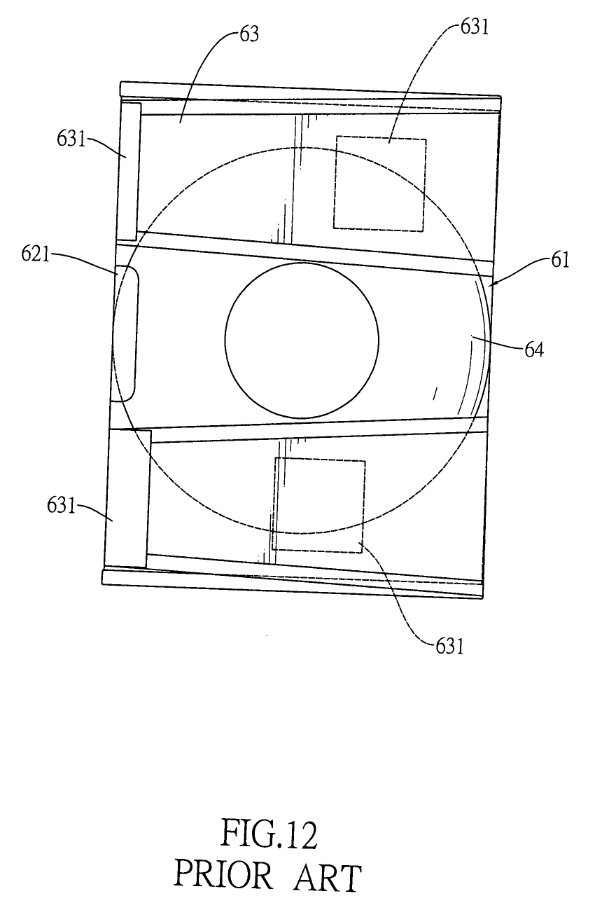

FIG. 13 is a front view of a variation of a conventional knee support; and

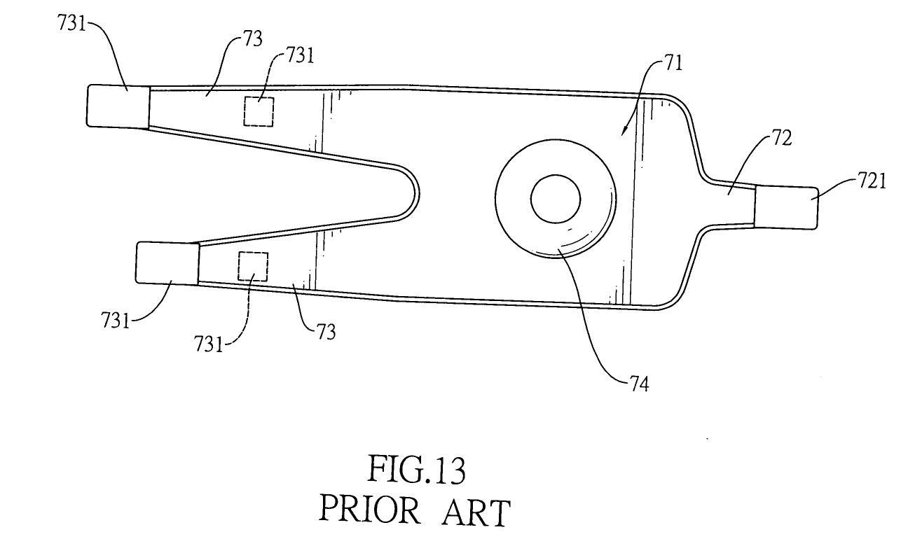

FIG. 14 is a front view of the knee support in FIG. 13, shown fastened, with a ring shown in phantom lines.

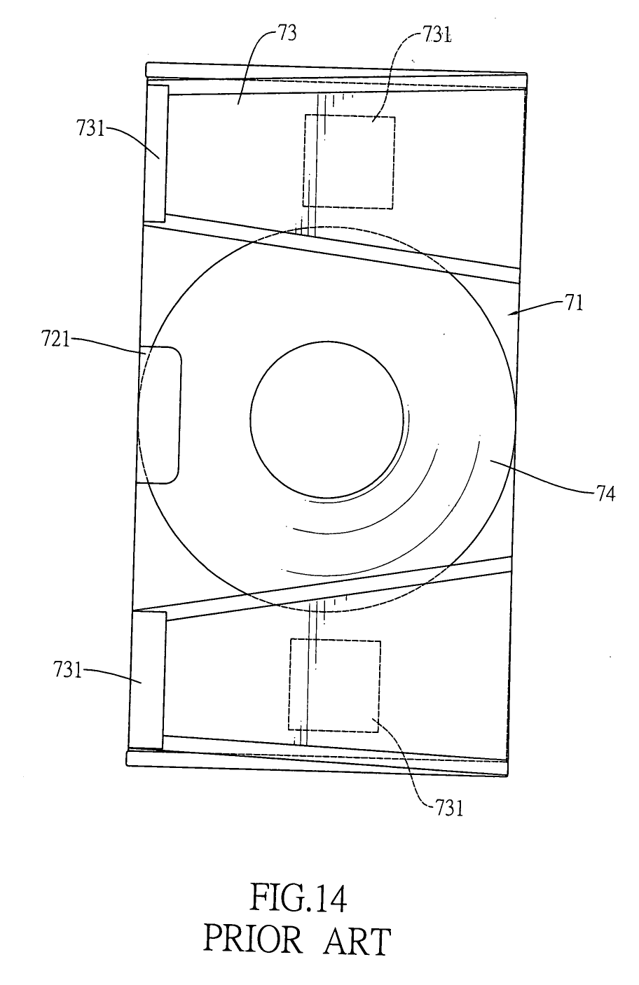

### DETAILED DESCRIPTION OF THE PREFERRED EMBODIMENTS

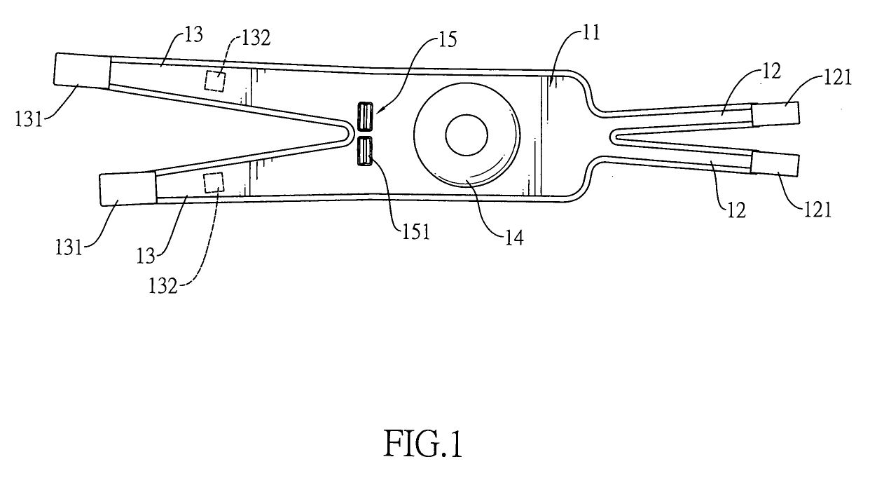

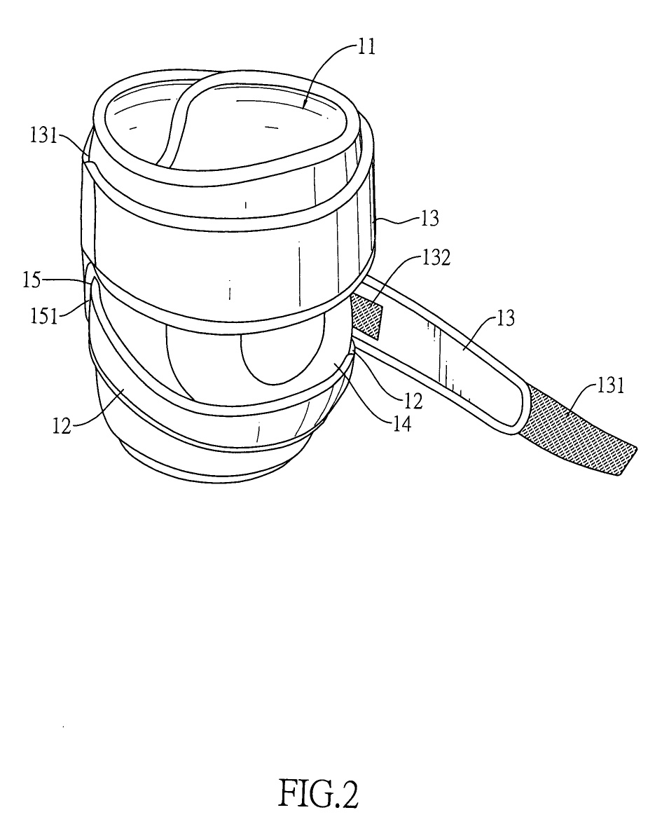

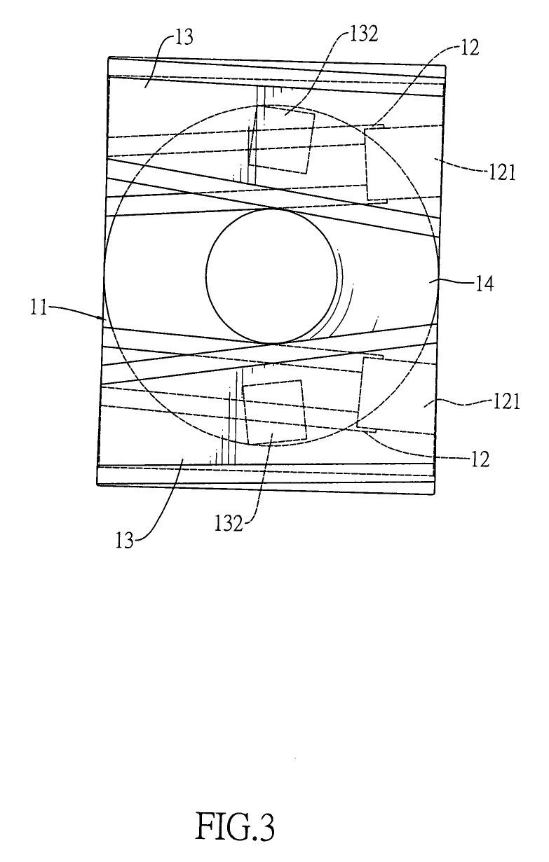

With reference to FIGS. 1, 2 and 3, a first embodiment of a knee support in accordance with the present invention comprises a body (11), a ring (14), at least one fastening strap (12), two securing straps (13) and a gap.

The body (11) is a resilient band having a width tied around a knee having a front and a patella to provide support and protection and comprises a fastening end, a securing end, an outer surface, an inner surface, a width and at least one hole (15). The outer surface is fluffy.

The at least one hole (15) of the body (11) is defined through the body (11) near the securing end and each hole (15). Each hole (15) has an inner edge and an lip (151). Each lip (151) is resilient and attached to the inner edge and may be rubber.

The ring (14) is elastic and mounted through the body (11) between the securing end and the fastening end and has a central hole. The ring (14) may be attached to the outer surface or inner surface of the body (11) and may be embedded inside the body (11). When the body (11) is fastened around the knee, the ring (14) is positioned at the front of the knee, the central hole being aligned with the patella for extra support and protection.

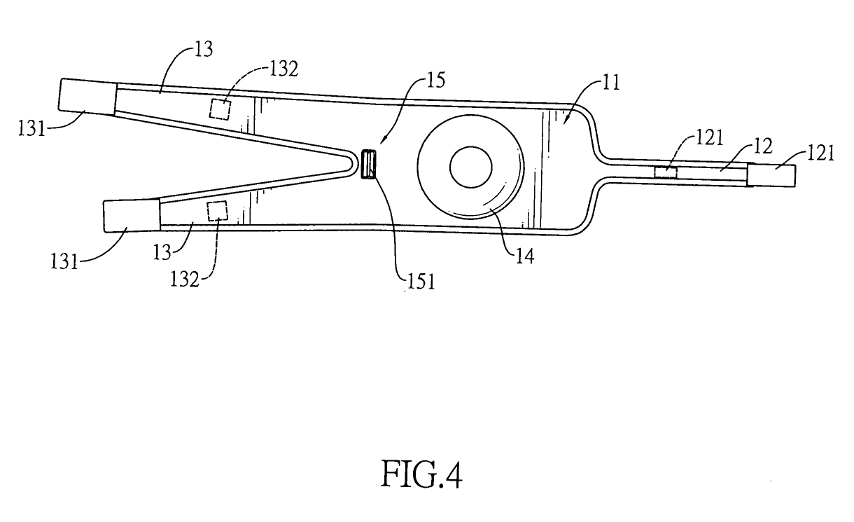

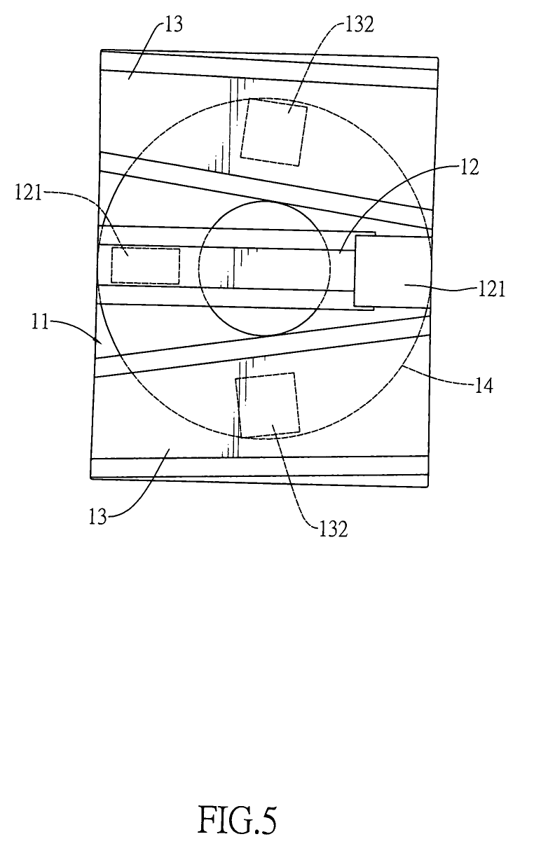

With further reference to FIGS. 4 and 5, the at least one fastening strap (12) is used to fasten the body (11) to the knee and to position the ring securely on the patella. The at least one fastening strap (12) extends from the fastening end. Each fastening strap (12) may extend through one hole (15) and be further held by the lips (151), is long enough to be wrapped around the body (11) and mounted over the ring (14) when the knee support is mounted wrapped around the knee. Each fastening strap (12) has a distal end, at least one fastener (121) and at least one auxiliary fastener (132). Since the at least one fastening strap (12) is used to press the ring (14) against the knee, the knee support still works effectively as the width of the body (11) is increased.

The fastener (121) is a hook portion of a hook-and-loop connector and is attached to the distal end of each fastening strap (12). Each fastening strap (12) is long enough to be wrapped around the knee and mounted over the ring (14) whilst the fastener (121) is attached to the outer surface. Since the fastening straps (12) hold the ring (14) tightly on the patella of the knee, the knee support in accordance with the present invention supports and protects the patella more effectively than a conventional knee support does.

The two securing straps (13) are used to hold the body (11) in position and secure the ring (14) and extend from the securing end. Each securing strap (13) has an end, an inner surface, a fastener (131) and at least one auxiliary fastener (132). The gap is defined between the two securing straps (13) allowing each fastening strap (12) to extend through the gap or the hole (15) and allow the knee to bend naturally. The securing straps (13) are wrapped around the knee and hold the ring (14) to the patella of the knee. Each fastener (131) is a hook portion of a hook-and-loop connector and is mounted on the inner surface of the end of the securing strap (13). Each auxiliary fastener (132) is a hook portion of a hook-and-loop connector and is mounted on the inner surface of the securing strap (13).

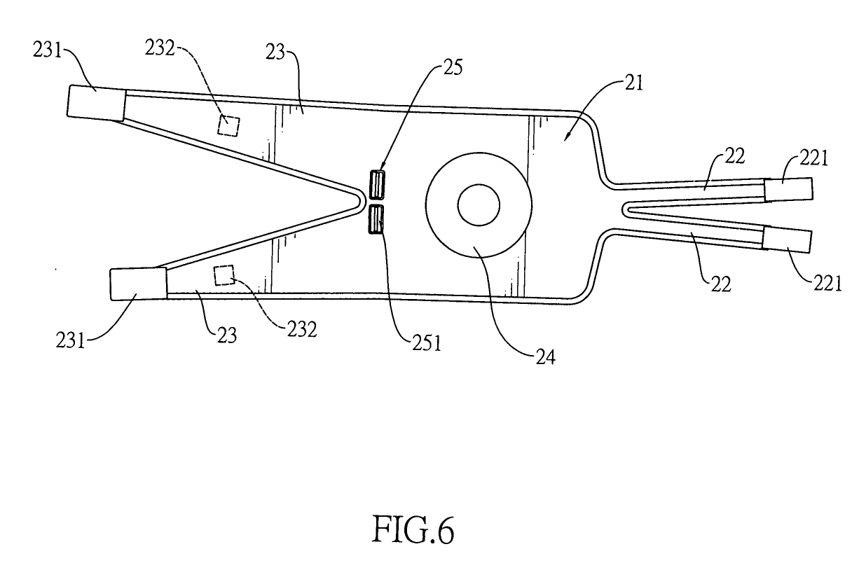

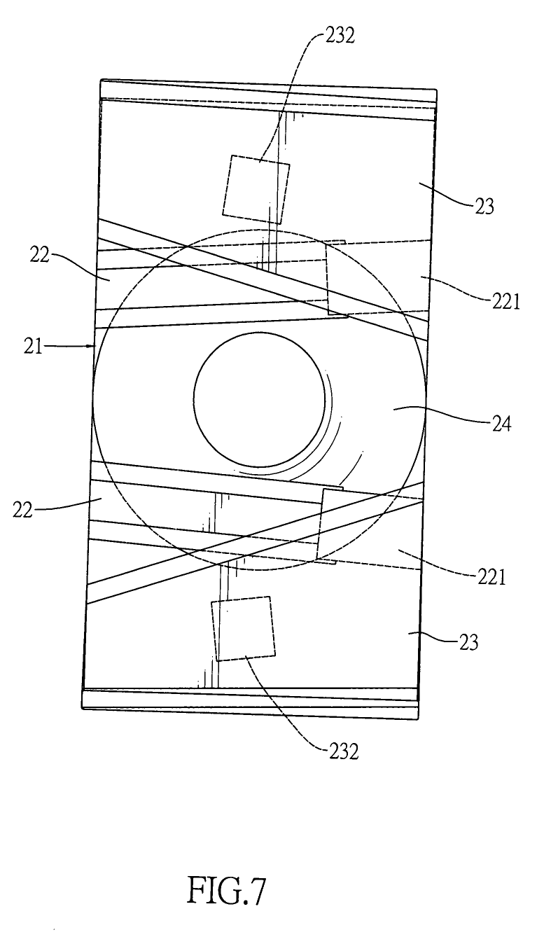

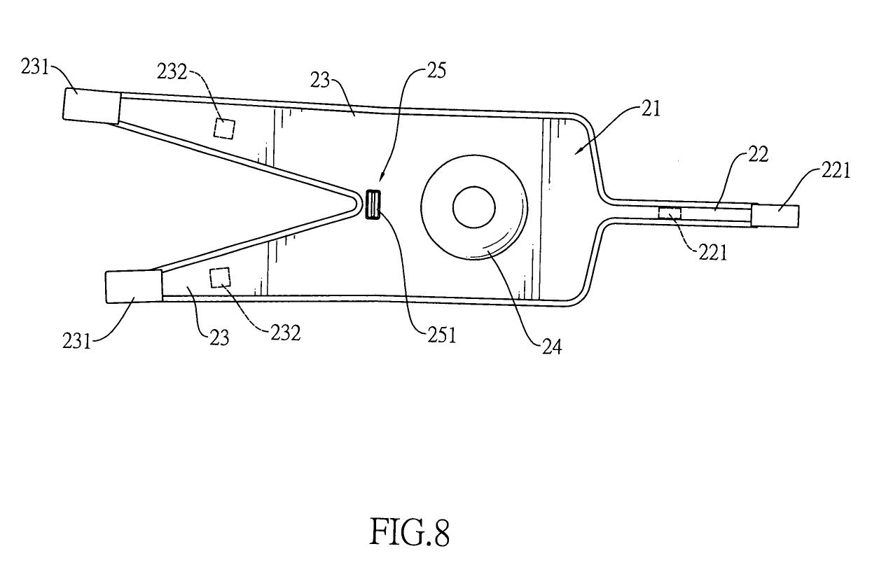

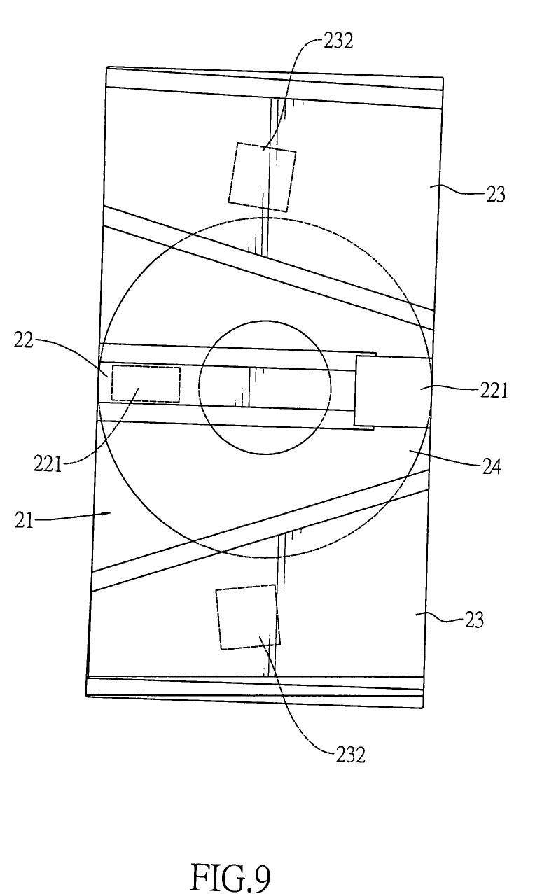

With further reference to FIGS. 6 to 9, a second embodiment of the knee support in accordance with the present invention is structurally similar to the first embodiment and has a ring (24), at least one fastening strap (22), two securing straps (23) and a body (21) being a resilient band having a width larger than that of the first embodiment enabling the second embodiment to be better adapted to a knee of a taller or stronger user. The body (21) comprises a fastening end, a securing end, and a fluffy outer surface.

The ring (24) is elastic and mounted through the body (21) between the fastening end and the securing end and has a central hole.

The at least one fastening strap (22) extends from the fastening end and each fastening strap (22) is long enough to be wrapped around the body (21) and mounted over the ring (24) when the knee support is adapted to be wrapped around the knee to effectively secure the ring (24) to the patella of the knee and protect the patella. Each fastening strap (22) has a distal end and at least one fastener (221) being a hook portion of a hook-and-loop connector attached to the distal end.

The two securing straps (23) extend from the securing end and each securing strap (23) has an end, an inner surface and a fastener (231) being a hook portion of a hook-and-loop connector attached to the end of the securing strap (23).

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A knee support for a knee having a patella, the knee support comprising
a body being a resilient band having a width and comprising
a fastening end;
a securing end; and
an outer surface being fluffy;
a ring being elastic and mounted through the body between the fastening end and the securing end and having a central hole;
at least one fastening strap being extending from the fastening end and each fastening strap being long enough to be wrapped around the body and mounted over the ring when the knee support is adapted to be wrapped around the knee, and each fastening strap having
a distal end; and
at least one fastener being a hook portion of a hook-and-loop connector and being attached to the distal end;
two securing straps extending from the securing end and each securing strap having
an end;
an inner surface; and
a fastener being a hook portion of a hook-and-loop connector attached to the end of the securing strap; and
a gap being defined between the two securing straps.

2. The knee support as claimed in claim 1, wherein the knee support has two fastening straps.

3. The knee support as claimed in claim 1, wherein
the body further comprises at least one hole being defined through the body near the second end and having an inner edge; and
the at least one fastening strap extends respectively through the at least one hole of the body.

4. The knee support as claimed in claim 3, wherein
each one of the at least one hole further has lips being made of a rubber based material and being attached to the inner edge; and
each one of the at least one fastening strap is held by the lips in one hole.

5. The knee support as claimed in claim 3, wherein the knee support has two fastening straps.

6. The knee support as claimed in claim 4, wherein the knee support has two fastening straps.

7. The knee support as claimed in claim 1, wherein at least one of the securing straps further has at least one auxiliary fastener being attached to the inner surface of the securing strap and being a hook portion of a hook-and-loop connector.

8. The knee support as claimed in claim 1, wherein at least one of the at least one fastening strap further has at least one auxiliary fastener being attached to the inner surface of the securing strap and being a hook portion of a hook-and-loop connector.

9. The knee support as claimed in claim 1, wherein the ring is embedded inside the body.
